(19) United States
(12) Patent Application Publication
(10) Pub. No.: US 2007/0135515 A1
(43) Pub. Date: Jun. 14, 2007

(54) Method for producing sorbicillactone a

(76) Inventors: Gerhard Bringmann, Wurzburg (DE); Gerhard Lang, Kiel (DE); Tobias Gulder, Wurzburg (DE); Karsten Schaumann, Bremerhaven (DE); Werner E.G. Mueller, Semmelweisstrasse (DE); Sanja Perovic-Ottstadt, Ginsheim-Gustavsburg (DE); Rudiger Stohr, Kiel (DE); Jutta Wiese, Kiel (DE); Rolf Schmaljohann, Kiel (DE); Johannes Imhoff, Kiel (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/588,099

(22) PCT Filed: Jan. 31, 2005
(86) PCT No.: PCT/EP05/00923

§ 371 Date: Dec. 11, 2006

(30) Foreign Application Priority Data
Jan. 30, 2004 (DE) 10 2004 004 901.7

(51) Int. Cl. A61K 31/365; C12P 17/02; C12N 1/18

(52) U.S. Cl. 514/450; 435/123; 435/254.5

(57) ABSTRACT

Methods for an improved culturing of the fungus Penicillium chrysogenum, in particular strain KIP 3201, for an optimised production of the anti-tumour natural compound sorbicillactone A (2), and derivatives thereof in large amounts 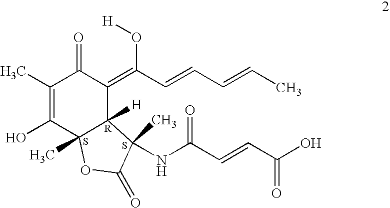 as well as optimised methods for the recovery and purification thereof from the fungal biomass and from the culture medium are described. Furthermore, the biological activity of sorbicillactone A (2), and studies regarding the genotoxicity of the compound are described.

The present invention relates to a method for the optimised production of the biologically active compound sorbicillactone A and derivatives thereof by culturing Penicillium chrysogenum, in particular strain KIP 3201. The invention furthermore relates to a method for purifying large amounts of sorbicillactone A and derivatives thereof from the culture medium and the fungal biomass as well as the use of sorbicillactone A for the treatment of diseases and infections.

### BACKGROUND OF THE INVENTION

The search for new biologically active compounds mainly takes place following two pathways. The isolation of active natural compounds from biological systems on the one hand, as well as the synthesis of novel compounds, partially inspired by secondary metabolites from the nature, on the other. With respect to this, the examination of the metabolites of macro- and microorganisms in habitats that are difficult to access and extreme is of particular interest. Thus, for example, marine eukaryontic organisms, specifically sponges, represent a rich source of bioactive substances (Sarma A S, Daum T, Müller W E G (1993) Secondary metabolites from marine sponges. Akademie gemeinnutziger Wissenschaften zu Erfurt, Ullstein-Mosby Verlag, Berlin). The reason for this is the sessile way of living of these organisms. They are dependent from the transport of their nutrients to the spot, floating particles (algae, bacteria or fungi), in the water. These particles are whirled in with a continuous stream of water. This leads to an increased danger of diseases caused by bacterial or fungal infections. Due to this reason, sponges are dependent from efficient defence mechanisms for a protection against such infections by biologically active secondary metabolites. Nevertheless, it often remains unclear, whether these substances are formed by the sponge itself or by one of the numerous microorganisms that are associated with the sponge. It is therefore of particular interest, to isolate these microorganisms living in the sponge, and to study their spectrum of metabolites. Therefore, it is a main goal of research to develop methods that allow to grow these microorganisms in culture in order to provide their biologically active secondary metabolites in sufficient amounts.

Today, it can be assumed that only about 5% of the microorganisms that are occurring in marine eukaryotic organisms can be cultured. Thus, it can be assumed that their potential is still largely unexploited. For a successful and sustainable use of these microorganisms as sources of novel pharmaceutics, it is important to develop culture methods that are characterised in that the production of the desired metabolites is increased as much as possible, while the formation of the unwanted side products is largely inhibited.

The spectrum of use for a commercial exploitation of the bioactive secondary metabolites is broad and reaches from the treatment of neurodegenerative diseases, bacterial, viral, and fungal infections to a therapy of tumours.

In doing so, it is also intensively sought for those bioactive substances that show a high specificity against defined tumours, and, at the same time, reduce opportunistic infections, e.g. by viruses.

The bioactive substance sorbicillactone A and the derivatives thereof were recently isolated from a strain of Penicillium that was isolated from a marine sponge of the genus Ircinia (DE 102 38 257.3). It was thereby found that sorbicillactone A and derivatives thereof have unexpected prominent anti-tumour and antiviral as well as anti-inflammatory characteristics. Until now, an upscaling of the method used for the production of this substance and their derivatives so far was not possible. Nevertheless, the reliable production of large amounts of sorbicillactone A and derivatives thereof is urgently needed for the promising use in the treatment of diseases, such as, for example, cancer and inflammations.

It is therefore the object of the present invention to provide a method for the optimised production of the natural compound sorbicillactone A and derivatives thereof, as well as for the extraction of sorbicillactone A and derivatives thereof from the culture medium and the fungal biomass, and for purifying large amounts of sorbicillactone A and derivatives thereof.

According to the invention, this object, first, is solved by providing a method that comprises the following steps:

a) culturing a fungus of the genus Penicillium at 20-25° C. in a suitable growth medium at a salt concentration of 2-5%, until the formation of a compact surface mycelium,

b) Increasing the temperature to 28-35° C. and further incubation for 5-10 days,

c) Separating the culture broth from the mycelium, and

d) Extracting sorbicillactone A and derivatives thereof from the culture medium, and, optionally,

e) Underlaying of the mycelium with fresh medium with a reduced salt concentration of 0.5-1.5%, and incubation at 28-35° C. for 3-8 days,

f) Repeating step c) and d), and optionally,

g) Repeating steps e) to f, and

h) Extracting sorbicillactone A and derivatives thereof from the culture medium and/or the mycelia.

According to the invention, this thus represents a method, wherein the culturing of the fungus takes place under particular conditions, leading to an increased yield and concomitant increased production rates. Thus, the growth and production of sorbicillactone A as well as precursors or derivatives thereof are controlled by the variation of the culturing conditions, and, for example, initiated and stimulated by the addition of NaCl. The production is particularly accelerated, since in step-wise processes the conditions for optimal growth and optimal production conditions for the production are successively realised, e.g. by a change of the incubation temperature of, for example, 20-25° C. to 28-35° C., and by a change of the salt concentration from 2-5% to 0.5-1.5%. At other incubation temperatures, no production at all or only low production of sorbicillactone A and/or derivatives thereof was found. Surprisingly, the method of the present invention thus reliably provides large amounts of sorbicillactone A and derivatives thereof.

Preferred according to the invention is a method, wherein the fungus Penicillium chrysogenum, in particular the strain KIP 3201, is used for the production.

A further aspect of the present invention represents a method that is characterised in that an increase of the yield of sorbicillactone A is achieved by modifying the growth media through a variation of the substrate and substrate concentrations, such as, for example, pyruvate, glutamate, proline, acetate, but also of sorbicilline and other biosynthetic precursors of sorbicillactone A.

A preferred embodiment represents a method, wherein the production of sorbicillactone A and derivatives thereof takes places in a flat bed method. A production of sorbicillactone A and derivatives thereof can furthermore be accelerated by separating the liquid phase from the cells at suitable points in time, and again stimulating the cells being supplied with fresh medium to a production. Suitable times are, for example, 3-10 days.

Preferred is a type of the inoculum in form of a solid state-bound form of the fungus, the material thereof is provided for inoculation on floatable solid states, e.g. grains or styrofoam globes.

Furthermore preferred is a method, wherein a sedimentation of the surface fungal mycelium is avoided. This can be achieved by integrating a carrier device for a stabilisation of the surface mycelium into the culture vessel. In doing so, a suitable form of a carrier device is represented, for example, by a mesh.

A preferred method is characterized in that the sorbicillactone A and derivatives thereof as produced can be bound immediately from the culture media to a solid exchanger. Then, a purification takes place in additional steps from this form as bound. In doing so, the elution from the exchanger can occur with organic solvents, such as methanol, ethanol, ethyl acetate, heptane or acetonitrile. In this manner, sorbicillactone A and derivatives thereof can be obtained in highly enriched form from the exchanger.

An additional preferred method is characterized in that the sorbicillactone A and derivatives thereof as produced from the fungal mycelium being separated from the culture medium are extracted by the addition of organic solvents. In doing so, preferably ethyl acetate is used.

An additional aspect of the present invention represents a method which is characterised in that a further extraction of the crude extracts is performed. In doing so, the crude extracts can be acidified, and subsequently sorbicillactone A and derivatives thereof can be extracted by means of organic solvents. In doing so, ethyl acetate is also preferably used.

It is furthermore advantageous to perform an optimised purification of the extracts by means of Fast Centrifugal Partitioning Chromatography (FCPC). Furthermore, an optimised purification of the extracts by means of gel chromatography, e.g. a Sephadex LH-20, is preferred. In doing so, different organic solvents for the elution of sorbicillactone A and derivatives thereof can be used.

A preferred aspect of the present invention is a method that is characterised in that it comprises a derivatisation of sorbicillactone A to sorbicillactone-A-methylester.

It was found that neuronal cells that were incubated with sorbicillactone A showed a strong decrease of the intracellular calcium level upon the addition of L-glutamic acid and serotonin (5-HT). L-Glutamic acid and serotonin are etiologically involved as neurotransmitters in a series of neurodegenerative diseases. Due to these characteristics, sorbicillactone A or derivatives thereof could be used in the treatment of neurodegenerative diseases, and the symptom-complexes related therewith.

Furthermore, the genotoxicity of sorbicillactone A was tested on L5178Y-mouse lymphoma cells (ATCC CRL 1722). It was found that in cells that were incubated with 1, 3, and 10 μg/mL sorbicillactone A no DNA-strand scissions were induced. In contrast, a significant increase of the portion of the DNA-strand scissions were found after 24 hours incubation at a concentration of 30 μg/mL of sorbicillactone A. Due to this property, it advantageous to use sorbicillactone A or derivatives thereof in the treatment of leukaemia.

In addition, sorbicillactone A induces apoptosis in L5178Y-cells after 4 hours of incubation at a concentration of 10 and 30 μg/mL. Due to these characteristics, the use of sorbicillactone A or derivatives thereof in the treatment of leukaemia is preferred.

Furthermore, sorbicillactone A or derivatives thereof can be used in the treatment of viral infections. Details regarding this are described in the examples of DE 102 38 257.3.

A further aspect of the present invention relates to a method for producing a pharmaceutical composition, wherein sorbicillactone A or derivatives thereof can be formulated together with suitable pharmaceutically acceptable auxiliary compounds and additives. Regarding this, preferred are pharmaceutical compositions, wherein sorbicillactone A or derivatives thereof are present in such an amount, that a concentration range of between 0.3 and 30 μg/ml is present during the treatment in vivo.

A further aspect of the present invention relates to the fungal strain of the genus Penicillium chrysogenum KIP 3201. It was deposited on Jan. 14, 2004 at the German Collection of Microorganisms and Cell Cultures GmbH under the number DSM 16137.

In the context of the present inventions, a “derivative” shall be a compound that is derived from the general formula 1, which, for example, is substituted by different radical groups as defined for R₁ to R₄ and X or Y, as well as mixtures of several of these compounds, which, for example, can be processed into a “personalised” medicament that is adjusted with respect to the disease to be treated and/or the patient on the basis of diagnostic data or data regarding the success or progress of the treatment, respectively. A derivative shall also mean a compound of the class of sorbicillactone A that can be isolated from (e.g.) marine organisms that are different from the ones as (exemplarily) mentioned here.

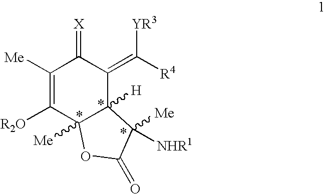

In the following, the present invention shall now be described by the following examples with reference to the enclosed figures, nevertheless, without being limited thereto.

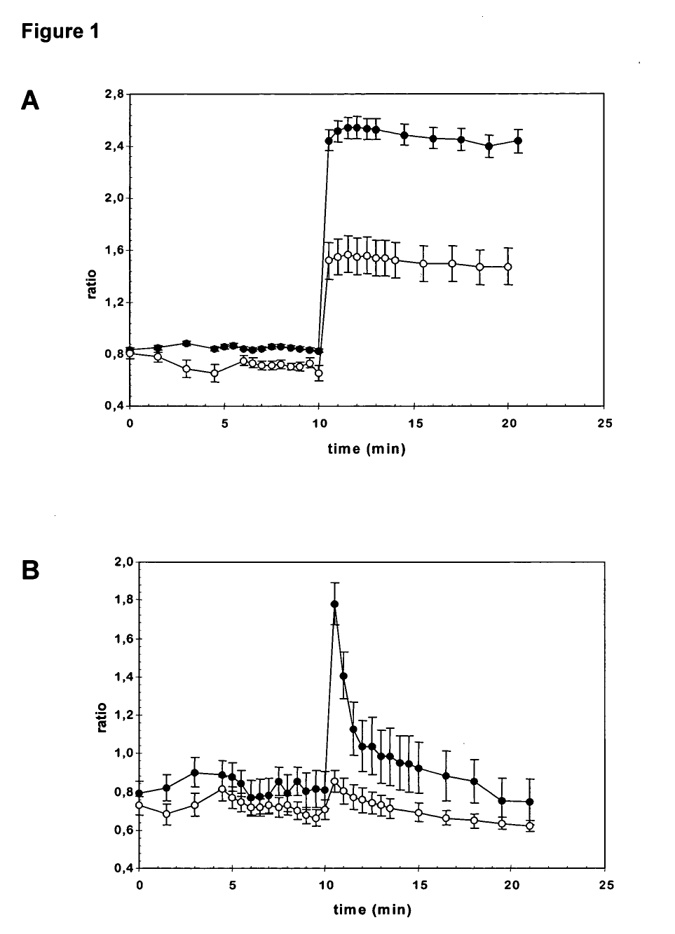

FIG. 1 shows the effect of sorbicillactone A on the intracellular calcium level of neuronal cells.

FIG. 1A shows the treatment of neurons with 200 μM L-glutamic acid (L-Glu) and 2.5 mM Ca²⁺ (●), wherein the neurons, that were treated with 10 μg/mL sorbicillactone A (∘), were pre-incubated 5 min before the addition of 200 μM L-Glu and 2.5 mM CaCl₂ (at time 10 min). The change in the kinetic was measured continuously for about 20 min. Both the mean value (n=19) as well as the standard deviation (±SE) were determined, respectively.

FIG. 1B shows the treatment of neurons with 200 μM serotonin (5-HT) and 2.5 mM Ca²⁺ (●), wherein the neurons, that were treated with 10 μg/mL sorbicillactone A (∘), were pre-incubated 5 min before the addition of 200 μM 5-HT and 2.5 mM CaCl₂ (at time 10 min). The change in the kinetic was measured continuously for about 20 min. Both the mean value (n=21) as well as the standard deviation (±SE) were determined, respectively.

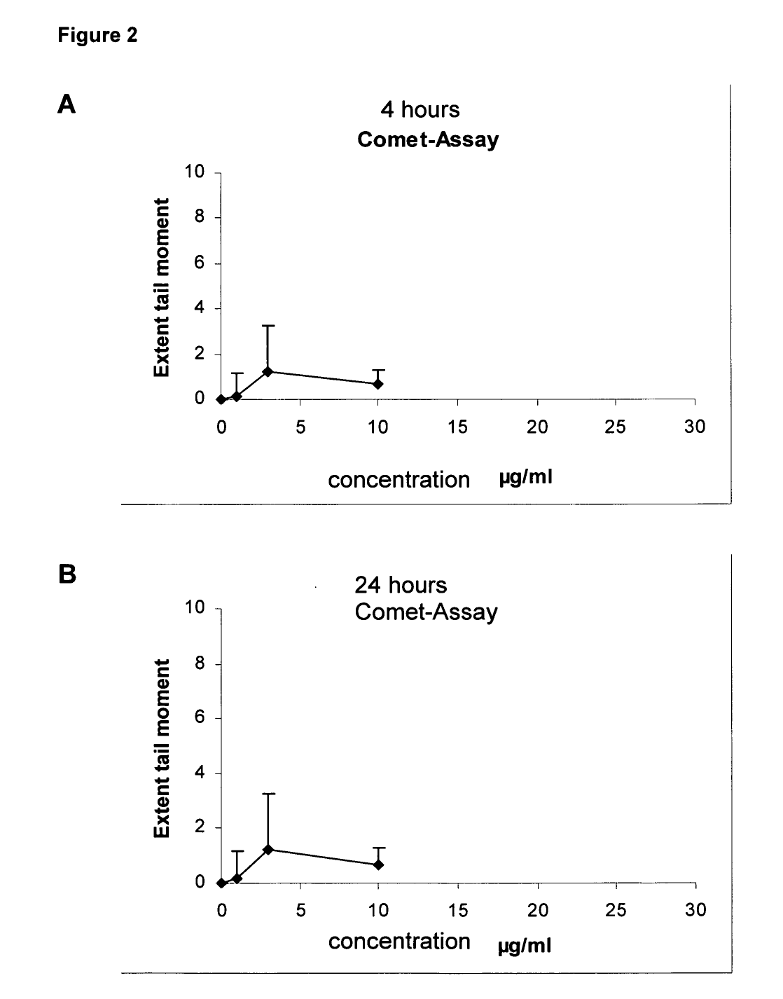

FIG. 2 shows the results of the “Comet-Assay” following the incubation of L5178Y-mouse lymphoma cells (ATCC CRL 1722) with 1, 3, and 10 μg/mL sorbicillactone A. FIG. 2A shows the results after 4 hours of incubation. FIG. 2B shows the results after 24 hours incubation. Both the mean value (A: n=25, B: n=29) and the standard deviation (±SD) were determined, respectively.

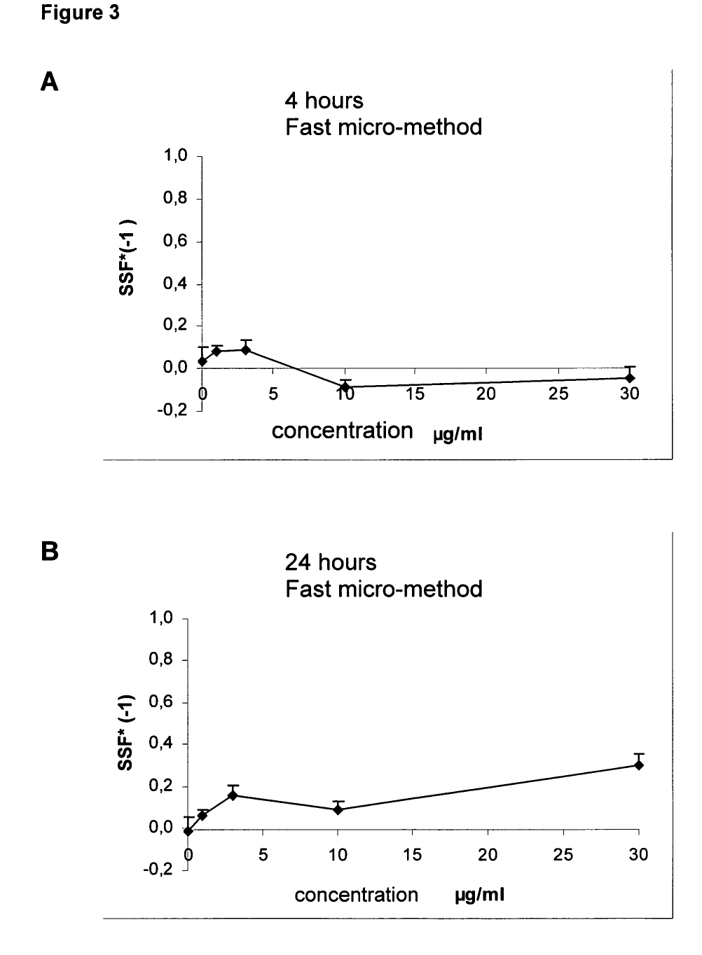

FIG. 3 shows the results of the “Fast Microassay” following the incubation of L5178Y-mouse lymphoma cells (ATCC CRL 1722) with 1, 3, 10, and 30 μg/mL sorbicillactone A. FIG. 3A shows the results after 4 hours of incubation. FIG. 3B shows the results after 24 hours of incubation. Both the mean value (A: n=5, B: n=7) and the standard deviation (±SD) were determined, respectively.

### EXAMPLE 1

### Culturing of the Fungus Penicillium chrysogenum in Salt-Containing Media for an Optimised Production of Sorbicillactone A

For optimising the production of sorbicillactone A, media and culture conditions were established that, amongst others, differ from the ones that are commonly used in that the cultivation takes place at increased salt contents and temperatures that are unusual for this organism.

Spore-suspensions are used as inoculum for the culture that have been generated under standard conditions, which were first grown on agar plates with Wickerham-medium with 1.5% bactoagar at room temperature for 14 days. The spores were suspended in a solution of seawater (34‰) and glycerol (2:1), adjusted to a standard titre, portioned, and frozen in suitable portions, and stored at −20° C. The material for inoculation for large-scale preparations of mass cultures is prepared in that grains are sterilized through autoclaving, supplemented with medium, and inoculated with fungal spores. After incubation for 14 days at 20° C. the grains are dried, and stored until use at room temperature. The culture media are inoculated with these spore preparations.

For the production, a modified version of the Wickerham-medium in the following composition is used: 3 g yeast extract, 6 g malt extract, 5 g peptone, 10 g glucose, 25 g NaCl in 1000 mL aqua dest. The pH is adjusted to 5.5.

The medium is added into culture vessels until a filling of 4 cm, autoclaved (20 min. at 121° C.), after cooling inoculated with spore suspension, and incubated for 7 days at 22° C. until the formation of a compact surface mycelium. Then, the temperature is increased to 31° C., and incubation continues for an additional 7 days. Then, the culture broth, which contains the major fraction of the sorbicillactone (1) as produced, is separated from the mycelium, and is further processed for the recovery of the substance. The mycelium is again underlayed with the same amount of fresh medium of a slightly modified composition. The medium as mentioned above is used, but with 5 g NaCl, 15 g glucose per litre, and without malt extract. Before the addition, the medium is warmed to the incubation temperature, and the mycelia are incubated for 5 days. Then, the same procedure is repeated. Sorbicillactone A (1) is extracted from the culture broths and the mycelia according to separate methods.

### EXAMPLE 2

### Extraction of Sorbicillactone A from the Fungal Biomass

The fungal mycelium is separated from the culture medium with a finely woven mesh, supplemented with 3 mL ethyl acetate per gram of biomass, and extracted. The extracts are filtered and concentrated in a rotating evaporator. The concentrate is stored at 5° C. or −20° C., respectively, until further purification.

### EXAMPLE 3

### Extraction of Sorbicillactone A from the Culture Medium

To the culture medium as separated from the mycelium 100 g of an exchange resin, e.g. XAD-16, are added per litre under slight stirring. Following the loading with the substances from the medium, the XAD-16 filtered off and subsequently extracted with methanol/water (1:1) and methanol. The extract is concentrated in a rotating evaporator, and the methanol-free concentrate is stored at 5° C. or −20° C., respectively, until further purification.

### EXAMPLE 4

### Determination of the Content of Sorbicillactone A by HPLC-UV

The content-determination takes place on an analytical HPLC with a diode-array-detector (gradient: 80% A:20% B to 20% A:80% B in 20 min. with A=water+0.05% TFA, and B=acetonitrile+0.05% TFA) on a Waters symmetry-C-18 reversed-phase-column at a flow of 0.4 mL/min. The integration of the peak area is done by a wavelength of 370 nm, since the absorption of dihydrosorbicillactone A (3) is practically zero at this wavelength, and the error in the determination of the content is thus minimised. The determination of the concentration in the crude extracts occurs by diluting the respective extract with a mixture of methanol/water (1:1) by a factor of about 150, in the extracts of the different purification steps the dilution is by a factor of about 10. The determination of the content of sorbicillactone A (2), both in the crude extracts as well a in the individual extracts of the different purification steps occurs with the aid of a calibration line from measurements with samples of differently concentrated solutions of sorbicillactone A (2) in methanol.

### EXAMPLE 5

### Purification Of the Crude Extract as Obtained from the Culture Medium

The aqueous crude extract of the fungal cultures of Penicillium chrysogenum is neutrally extracted with ethyl acetate for a separation of unwanted side products, such as, for example, meleagrin. The ethyl acetate-phase as obtained is discarded. The aqueous phase is acidified with phosphoric acid (pH=2) and exhaustively extracted with ethyl acetate. Sorbicillactone A can be transferred in a practically quantitative manner into the ethyl acetate-phase of the acidic extraction. The extract as obtained already contains an amount of up to 50% of its mass of sorbicillactone A after concentration in vacuo.

### EXAMPLE 6

### Further Purification of the Extract by Fast Centrifugal Partitioning Chromatography (FCPC)

The further purification of the extract occurs with an additional liquid-liquid-chromatographic step. In doing so, the novel method of the so-called Fast Centrifugal Partitioning Chromatography (FCPC) is employed. In this method, just like in common liquid-liquid-chromatographic methods, such as, for example, the High Speed Countercurrent Chromatography (HSCCC), a 2-phase mixture of solvents is used. In doing so, optionally the upper or the lower phase can be used as stationary phase. In contrast to HSCCC, FCPC does not use a capillary coil, but a rotor that is provided with several hundreds of separation chambers. In these chambers, which are arranged directly one after the other, the separation of substances that are contained in the extract takes place between the mobile and stationary phase.

During the separation, the system is brought into rapid rotation (1200-1400 rpm). In doing so, on the one hand, in accordance with the direction of flow, the desired phase is retained in the rotor of the FCPC, and, on the other hand, the separation of both phases is accelerated by the centrifugal force. This allows for the use of high velocities of flow, and thus for the throughput of large amounts of substances within a short time. The separation coefficient K of the desired substance between the two phases should lie in the range of between 0.7 and 4.5. At a smaller K, the substance elutes too quickly, and thus no separation occurs at all. In contrast, at a higher K, the retention time becomes too long for the quick purification of large amounts of extract.

In case of sorbicillactone A, the use of a mixture of solvents of heptane/ethyl acetate/methanol/water (42%/58%/42%/58%) at a flow of 6 to 7 mL per min., a number of revolutions of 1200 rpm, and the use of the upper phase as a stationary phase is particularly advantageous. In addition, 1 mL of concentrated phosphoric acid is added per litre of mixture of solvents. Upon use of a 200 mL-FCPC-rotor, a purification of up to 1.5 g extract per run is possible. With these settings, only 90 min are required per separation, including the time periods for preparation and washing. The sorbicillactone-containing fractions are separated from the organic solvents, and the remaining aqueous-acidic phase is exhaustively extracted with ethyl acetate. After concentration in vacuo, extracts with a mass-content of sorbicillactone A of up to 70% can be obtained.

### EXAMPLE 7

### Recovery of Pure Sorbicillactone A by Means of Gel Chromatography

The most difficult step in the recovery of pure sorbicillactone A (2) is the separation of the structurally very similar, but five-fold less active compound dihydrosorbicillactone A (3).

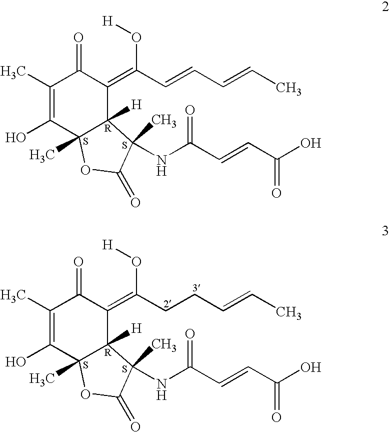

FIG. 1. Structures of Sorbicillactone A (2) and Dihydrosorbicillactone A (3)

Due to the fact that both compounds only differ in the grade of saturation at the C-2′ and C-3′ in the sorbyl side chain, the physical characteristics of the molecules, such as mass or polarity, that can be used during the separation of mixtures of compounds, are nearly identical. Nevertheless, the separation can be realised by gel chromatography on Sephadex-LH-20-material with methanol as eluent. However, due to the large similarity of the molecules, the use of a very long column-system (longer or equal to 6 m) is required. This column-system, as an MPLC-system, is provided with solvent by a pump. The velocity of flow of the eluent is about 10 mL per min. In doing so, both lactones 2 and 3 are sufficiently resolved one from the other, wherein 3 eluates faster. Per separation process, about 70% of the contaminated sorbicillactone A (2) as applied can be obtained in clean fractions. The mixed fractions that are contaminated with 3 can be purified further without problems by a restarted chromatography on Sephadex LH-20.

### EXAMPLE 8

### Determination of the Percentage Ratio of Sorbicillactone A (2) and Dihydrosorbicillactone A (3) in the Extracts by HPLC-UV

For determining the percentage ratio of sorbicillactone A (2) and dihydrosorbicillactone A (3) in the crude extracts, first, a sample of the extract is diluted with a mixture of methanol/water. The sample is examined on an analytical HPLC with diode-array-detector under isocratic conditions (water/acetonitrile/TFA=70/30/0.05%) on a Waters Symmetry-C-18 reversed-phase-column at a flow of 0.4 mL/min. With this, one obtains a separation that is suitable for the integration of the peak areas of the compounds. In doing so, the integration of the peak areas is performed at a wavelength of 220 nm, since the absorption of both lactones (2, 3) at this wavelength is approximately identical. This method can also be employed for the examination of the purity of the fractions from gel chromatography.

### EXAMPLE 9

### Determination of the Purity of the Fractions from the Gel Chromatography by Means of UV-Absorption Experiments

In order to accelerate the analytics of the fractions from the final purification step, a method was sought for that would do without chromatographic methods. In doing so, it was found to be feasible to record the UV-absorption of the respective fractions both at 300 nm as well as at 430 nm, and to use the quotient from both values as measured for checking the purity. The undesired contamination dihydrosorbicillactone A (3) eluted before sorbicillactone A (2), and, just like sorbicillactone A (2), strongly absorbed at 300 nm, but did nearly not do so at a wavelength of 430 nm. Upon reaching a constant quotient from both values as measured, it can therefore be assumed that the fractions contain only pure sorbicillactone A (2). These results could be confirmed with control measurements under the conditions as described in example 8. Thus, the measuring time for the analytics of all fractions of a separation can be reduced from several hours with the HPLC-analytics to only a few minutes.

### EXAMPLE 10

### Derivatisation of Sorbicillactone A (2) to its Methyl Ester 4

The derivatisation of sorbicillactone A (2) to its methyl ester 4 was interesting, on the one hand, since the compound was suitable as an internal standard for the determination of the concentration in e.g. blood serum, and, on the other hand for studying the structure-activity relations. 30 mg sorbicillactone A (2) were dissolved in 5 mL of methanol, and supplemented with 200 μL of concentrated sulphuric acid. After 6 hours of stirring at room temperature, 100 mL water was added, and the mixture was extracted twofold with 100 mL of ethyl acetate. After evaporation of the organic phases in vacuo the residue was purified by preparative HPLC. 18.6 mg of a yellow amorphous substance were thus obtained.

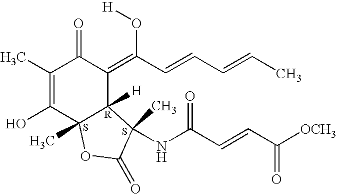

Physical and Spectroscopic Data of Sorbicillactone-A-Methyl Ester (4):

The compound is present as an amorphous yellow solid.

Melting temp. 166-170° C. (THF).

[α]_{D}²⁰=−558° (c=0.2 in methanol).

CD (c=0.2 in methanol): Δε₂₀₈ +11.1, Δε₂₃₁ −12.6, Δε₂₇₈ +12.5, Δε₃₇₀ −13.0.

IR (KBr): ν=3333 (br.), 2931 (w), 1783 (m), 1730 (m), 1681 (s), 1612 (s), 1552 (s), 1442 (m), 1415 (m), 1384 (m), 1350 (s), 1310 (s), 1198 (m), 1176 (m), 1065 (m) cm⁻¹.

MS (ESI, positive): m/z 432 [M+H]⁺.

TABLE 1
NMR-data of sorbicillactone-A-methyl ester (1) in THF-d₈

| Position | ¹³C [ppm] | ¹H [ppm] | | HMBC |
|---|---|---|---|---|
| 1 | 99.5 | | | |
| 2 | 192.0 | | | |
| 3 | 110.8 | | | |
| 4 | 166.7 | | | |
| 5 | 81.1 | | | |
| 6 | 53.2 | 3.47 | s | 1, 2, 4, 5, 7, 9, 11, 1′ |
| 7 | ˜23.5 | 1.55 | s | 4, 5, 6 |
| 8 | 7.3 | 1.52 | s | 2, 3, 4 |
| 9 | 60.0 | | | |
| 10 | 173.0 | | | |
| 11 | ˜26.0 | 1.42 | s | 6, 9, 10 |
| 1′ | 169.7 | | | |
| 2′ | 121.7 | 6.39 | d | 1′, 3′, 4′ |
| 3′ | 139.1 | 7.20 | dd | 1′, 3′, 5′ |
| 4′ | 131.9 | 6.28 | ddd | 2′, 6′ |
| 5′ | 137.0 | 6.09 | m | 3′, 6′ |
| 6′ | 18.6 | 1.82 | dd | 4′, 5′ |
| 1″ | 162.3 | | | |
| 2″ | 136.3 | 6.71 | d | 1″, 3″, 4″ |
| 3″ | 130.3 | 6.52 | d | 1″, 4″ |
| 4″ | 165.9 | | | |
| COOMe | 51.8 | 3.69 | s | 4″ |
| 1′-OH | | 16.60 | bs | |
| NH | | 7.66 | s | 9, 10, 11, 1″ |

### EXAMPLE 11

### Detection of Bioactivity of Sorbicillactone A

A) Measurements

Material: The same materials as described in Perovic et al. (1998, Mech. Ageing Dev. 101:1-19) were used. Fura-2-acetoxymethylester (Fura-2-AM) was obtained from Molecular Probes (Leiden, The Netherlands), Dulbecco's modified Eagle's Medium with 4.5 g/L glucose (DMEM/HG), L-glutamic acid (L-Glu), and serotonin (5-HT) from Sigma-Aldrich (Taufkirchen, Germany) and the antibodies mouse-anti-neurofilament (68 kDa) and mouse-anti-glial-fibrillary-acidic-protein (GFAP) from Roche Diagnostics (Mannheim, Germany). Sorbicillactone A of the batch: 2208/2a was used.

Primary neurons: The cortical cell culture was produced from the brains of 17-18 day-old rat embryos, according to a modified procedure [Freshney (1987) Culture of specific cell types. In: Culture of Animal Cells. A Manual of Basic Technique, A. R. Liss, New York, S. 257-288; Perovic et al. (1994) Eur. J. Pharmacol. (Mol. Pharmacol. Sec.) 288:27-33]. After the isolation, the cerebral-hemispheres were put into Hank's Balanced Salt Solution (HBSS) without Ca²⁺ and Mg²⁺, and the neuronal cells were subsequently dissociated in HBSS using 0.025% (w/v) trypsin (10 min.; 37° C.). The proteolytic reaction was stopped with 10% (v/v) foetal calf serum (FCS). The single-cell suspensions were centrifuged, and the resulting pellet was taken up into Dulbecco's modified Eagles's Medium with 4.5 g/L glucose (DMEM/HG) (contains 2 mM L-glutamine, 100 mU/L insulin, and 10% (v/v) FCS). The cells were plated in a chamber with poly-L-lysine (5 μg/mL, 300 μl/cm²) in a cell density of 2.0×10⁵ cells/cm². After two days the DMEM/HG/10%-FCS-medium was removed and replaced with DMEM/HG/serum-free medium. Two weeks after the isolation, the immune staining took place with anti-neurofilament (68 kDa) as a marker for neurons, and anti-GFAP as a marker for glial cells. The cultures contained >80% neurons; the residual 20% were GFAP-positive cells, mainly astrocytes (Ushijima et al. (1995) Eur. J. Neurosci. 7:1353-9). The neurons were held in an atmosphere of 95% air and 5% CO₂ at 37° C.

Loading of neurons with Fura-2-AM: The intracellular Ca²⁺-concentration ([Ca²⁺]ᵢ) was determined by fluorescence measurements. The ratio of the absorption of the Ca²⁺-indicator dye Fura-2-AM at 340 and 380 nm (Grynkiewicz et al. (1985) J. Biol. Chem. 260:3440-50) was decisive. The neurons were loaded with 6 μM Fura-2-AM in DMEM/HG/serum-free medium (supplemented with 1% (w/v) bovine serum albumin) at 37° C. for 60 min. After incubation, the cells were washed two times with medium, and further incubated for 45 min at 37° C. This incubation period is sufficient in order to load the neurons (inactive Fura-2-AM) and to hydrolyse the acetoxymethyl ester (active Fura-2).

Calcium-calibration curve: A calcium-calibration curve was produced according to the methods of Grynkiewicz et al. (1985, J. Biol. Chem. 260:3440-50). Fluorescence pictures were obtained for each buffer at 340 and 380 nm. The quotient from both fluorescence spectra (340/380 nm) was calculated, and depicted as a calibration curve. The quotient (340/380 nm [ratio-value]) of 1.0 corresponds to 228 nM [Ca²⁺]ᵢ.

Changes of the calcium level in neurons: In all experimental settings, the cells were first loaded with Fura-2-AM, and subsequently stimulated with different substances (in Locke's solution; 154 mM NaCl; 5.6 mM KCl; 3.6 mM NaHCO₃; 5.6 mM glucose, and 10 mM Hepes; pH 7.4; without CaCl₂). Sorbicillactone A was dissolved in a concentration of 10 mg/mL in 100% (v/v) DMSO, and stored at −20° C. In the first setting, the neurons were stimulated for 5 min with 0.1% (v/v) DMSO (control); after 10 min 200 μM L-glutamic acid (L-Glu), 200 μM serotonin (5-HT), and 2.5 mM CaCl₂ were added to the cells. In a second setting, the effect of L-Glu, 5-HT, and 2.5 mM CaCl₂ on the calcium level of pre-incubated neurons (each 5 min with 10 μg/mL sorbicillactone A) was tested. In all experiments, the [Ca²⁺]ᵢ-level was measured for at least 20-25 min.

For a determination of the [Ca²⁺]ᵢ, the cells were cultured on poly-L-lysin-coated borosilicate-object slides in the 4-chamber-system (Lab-Tek® Chamber Slide™ system; Nunc, Wiesbaden, Germany). The fluorescence measurements were performed with an inverted-stage-microscope (Olympus IX70) with apochromatic reflected light and the fluorescence objective UApo40X/340. The cells were illuminated with alternating light of the wavelength 340 and 380 nm with the aid of a computer-controlled narrow-band-interference filter in front of a 100-W-xenon-lamp. In addition, a 0.25-ND-filter was used at 380 nm. The fluorescence emissions at 510 nm were recorded with a CCD-camera (model C2400-87; Hamamatsu, Herrsching, Germany). The pictures were digitalised with computer-assistance with the imaging-system Argus 50, Hamamatsu, as 256×256 pixel with 8-bit-arrays. The fluorescence quotient 340/380 nm was determined by a division of the pairs of pictures.

Statistics: The results were interpreted by means of the paired “student's t-test” (Sachs (1984) Angewandte Statistik. Springer, Berlin).

B) Results

Effect of sorbicillactone A on the [Ca^{2+]}ᵢ-level in neuronal cells: The addition of 10 μg/mL sorbicillactone A showed nearly no effect on [Ca²⁺]ᵢ in neuronal cells.

Effect of L-glutamic acid on the [Ca²⁺]ᵢ-level in the presence or absence of sorbicillactone A in neuronal cells: Incubation of the cells with 200 μM L-glutamic acid and 2.5 mM Ca²⁺ (FIG. 1A) resulted in a strong increase of [Ca²⁺]ᵢ after 10 min. At the end of the measurements, the 340-/380-nm-values increased by 1.715±0.081 (307.1%). In doing so, the control was assumed to be 100%. A pre-incubation of the neurons with 10 μg/mL sorbicillactone A resulted in a reduction of the [Ca²⁺]ᵢ-level after the addition of 200 μM L-Glu and 2.5 mM CaCl₂. The reduction in the [Ca²⁺]ᵢ-level was significant and was 46.7% at 10 μg/mL (p<0.001, FIG. 1A).

Effect of serotonin on the [Ca²⁺]ᵢ-level in the presence or absence of sorbicillactone A in neuronal cells: The addition of 200 μM Serotonin (5-HT) and 2.5 mM Ca²⁺ to the cells (FIG. 1B) resulted in a strong increase of [Ca²⁺]ᵢ after 10 min. The 340-/380-nm-values increased by 0.971±0.112 (219.9%). In doing so, the control was j assumed to be 100%. The reduction of the free calcium concentration in neurons following pre-incubation with 10 μg/mL sorbicillactone A and subsequent incubation with 200 μM of 5-HT was significant; compared to the control the values were reduced by 77.6% (FIG. 1B).

C) Conclusions

It was found that cells that were incubated with sorbicillactone A, showed a strong decrease of the intracellular calcium level after the addition of L-glutamic acid and serotonin (5-HT). L-glutamic acid and serotonin as neurotransmitters are etiologically involved in a series of neurodegenerative diseases. Due to these characteristics, sorbicillactone A can be used in the treatment of the diseases as indicated, and the symptom-complexes that are associated therewith.

### EXAMPLE 12

### Detection of the Genotoxicity of Sorbicillactone A: “Comet-Assay”

Material: “Normal Melting Agarose” (NMA, Cat.-No. 840041) and “Low Melting Agarose” (LMA, Kat.-Nr. 870081) were obtained from Biozym (Hamburg, Germany), Triton x-100 from Fluka (Buchs S G, Schwitzerland), RPMI1640-medium, DMSO, and EDTA from Sigma-Aldrich (Taufkirchen, Germany), NaCl and Tris from Roth (Karlsruhe, Germany) and foetal calf serum (FCS) from Gibco (Karlsruhe, Germany). Sorbicillactone A of the batch: 2208/2a was used.

Cells: The genotoxicity of sorbicillactone A was tested on L5178Y mouse lymphoma cells (ATCC CRL 1722). As described (Müller et al., 1979, Cancer Res. 39:1102-1107), the cells were cultured in RPMI1640-medium with 10 mM Hepes, which was supplemented with 10% foetal calf serum (FCS). As a concentration of the inoculum, 10⁴ cells/mL was chosen. The cells were incubated with 1, 3, and 10 μg/mL sorbicillactone A for 4 and 24 hours. After the incubation, the cells were examined with the “Comet-Assay”.

Alkaline electrophoresis: The object slides (Superfrost) were cleaned with acetone. 1.0-percent “Normal Melting Agarose” (NMA) in 1×PBS was cooked, and subsequently about 600 μl of agarose were applied onto an object slide. The object slides were shortly (˜5 min) dried at 50° C., and stored over night at room temperature. Then, the 0.7-percent “Low Melting Agarose” (LMA) was prepared in distilled water. The second layer on the object slides consisted of 200 μl LMA. The object slides were incubated at 4° C. for 10 min. The 3^{rd} layer consisted of 10 μl of a cellular suspension (7×10⁴ cells/mL) plus 60 μl LMA. After incubation with sorbicillactone A, the cells were washed for one time with 1×PBS, and centrifuged at 800×g for 5 min at 4° C. The cell count was diluted to 7×10⁴ cells/mL. Subsequent to the application, the object slides were incubated for 10 min at 4° C. Before the lysis of the cells, the last LMA-layer (˜100 μl) was applied. The lysis of the cells took place in the dark by incubation in lysis solution (10 mM Tris, 100 mM EDTA, 2.5 M NaCl; pH=10) with 10% (v/v) DMSO and 1% (v/v) Triton x-100 for 1 hour at 4° C. The object slides were directly transferred from the lysis into the electrophoresis-chamber, and incubated for 20 min in the electrophoresis-buffer (30 mM NaOH, 1 mM EDTA; pH 13.8). The electrophoresis was constantly set at 0.75 V/cm (˜300 mA), and was performed under cooling on ice for 30 min. For neutralisation, the object slides were washed for 5 min with neutralisation buffer (400 mM Tris; pH 7.5) at room temperature, and subsequently dehydrated for 5 min. with 95% ethanol, air-dried in the dark. The object slides were stained with 60 μl of ethidium bromide-solution (20 μg/mL dist. water), photographed and interpreted. The values are given as extent tail moment (Bihari et al., 2002, Croatica Chemica Acta 75:793-804; Müller et al., 1979, Cancer Res. 39:1102-1107). A high number thus points to a high disintegration of the DNA in the cells. In normal cells, values around 0 can be found.

Results: The incubation of the L5178Y-cells with 1, 3, and 10 μg/mL sorbicillactone A did not exhibit significant changes of the extent tail moment after 4 hours (FIG. 2A). At these concentrations, sorbicillactone A does not induce DNA-strand scissions. Also after 24 hours incubation with sorbicillactone A, no significant changes of the extent tail moment in L5178Y-cells could be detected (FIG. 2B).

Conclusion: It was found that cells that were incubated with sorbicillactone A did not show a significant increase of the extent tail moment after the addition of 1, 3, and 10 μg/mL.

### EXAMPLE 13

### Detection of the Genotoxicity of Sorbicillactone A: “Fast Microassay”

Material: PicoGreen was obtained from Molecular Probes (Leiden, The Netherlands), RPMI1640-medium from Sigma-Aldrich (Taufkirchen, Germany), foetal calf serum (FKS) from Gibco (Karlsruhe, Germany), and black microtitre-plates (96-well-plates) from Nunc (Wiesbaden, Germany). Sorbicillactone A of the batch: 2208/2a was used.

Cells: The genotoxicity of sorbicillactone A was tested on L5178Y mouse-lymphoma cells (ATCC CRL 1722). As described (Müller et al., 1979, Cancer Res. 39:1102-1107), the cells were cultured in RPMI1640-medium supplemented with 10% foetal calf serum (FCS). 10⁴ cells/mL were chosen as the inoculum concentration. The cells were incubated for 4 and 24 hours with 1, 3, 10, and 30 μg/mL of sorbicillactone A (1). Following the incubation, the cells were examined with the “Fast Microassay”.

“Fast Microassay”: The method was performed according to a modified method (Batel et al., 1999, Anal. Biochem., 270:195-200). The cells were washed with 1×PBS for two times. After centrifugation, the pellet was taken up in 1×PBS and diluted to 10⁴ cells/mL. 25 μl each of the cell suspension (about 2.5×10⁴ cells per well) were pre-pipetted into black microtitre-plates (96-well-plates, Nunc, Wiesbaden). 25 μl 1×PBS were in the controls. Then, 25 μl of lysis solution (7 M urea, 0.1% (w/v) SDS, 0.2 M EDTA, pH=10.0) with PicoGreen (20 μl conc. PicoGreen per 1 mL of lysis solution) were pipetted into the microtitre-plate. Subsequently, the cells were lysed for 40 min in the dark at room temperature. After the incubation, the denaturation (unwinding) of the DNA occurs by the addition of freshly prepared alkaline solution. This is produced in that 20 mL 20 mM of EDTA, and 32 mL of 20 mM EDTA/100 mM NaOH are adjusted to a pH-value of 12.3. The measurement took place immediately in a fluoroscan at an excitation wavelength of 485 nm, and an emission wavelength of 520 nm, every 3 to 5 min, 20 min in total. The results were given as “strand scission factors” (SSF), and calculated following a time of denaturation (time of unwinding) of 20 min according to the following equation: SSF=log (% dsDNA-sample/% dsDNA-control).

Results: Effect of sorbicillactone A on the SSF-value in L5178Y-cells: The addition of 1, 3, 10, and 30 μg/mL of sorbicillactone A showed nearly no effect on the SSF-values in L5178Y-cells after 4 hours of incubation (FIG. 3A).

A significant increase of the SSF-values (portion of DNA-strand scissions) was observed after 24 hours upon the addition of 3 and 30 μg/mL (p<0.001) of sorbicillactone A (FIG. 3B). Incubation of the L5178Y-cells with 1 to 10 μg/mL of sorbicillactone A showed a slight, but no significant, increase of the SSF-values.

Conclusions: Sorbicillactone A in L5178Y-cells induced DNA-strand scissions after 24 hours of incubation at a concentration of 30 μg/mL. Due to these characteristics, sorbicillactone A can be used in the treatment of leukaemia.

### EXAMPLE 14

### Detection of the Apoptosis as Induced by Sorbicillactone A (Cell Death Detection ELISA Plus)

Material: RPMI1640-medium, Hepes buffered, was obtained from Sigma-Aldrich (Taufkirchen, Germany), foetal calf serum (FCS) from Gibco (Karlsruhe, Germany), and Cell Death Detection ELISA plus from Roche (Mannheim, Germany, Cat. No. 1774425). Sorbicillactone A of the batch: 2208/2a was used.

Incubation and preparation of the cells: The L5178Y-mouse lymphoma cells (ATCC CRL 1722)) were counted, and adjusted to a number of cells of 10⁴ cells/mL. The sorbicillactone-A- and dihydrosorbicillactone-A-stock solution (10 mg/mL in DMSO) was diluted with medium (RPMI/Hepes with 10% FCS) to concentrations of 60, 20, and 6 μg/mL. In a 96-well-culture plate, 100 μl cell suspension (˜10³ cells) were each pipetted to each of the 100 μl of the different dihydrosorbicillactone-A- and sorbicillactone-A-solutions. RPMI1640-medium without dihydrosorbicillactone and sorbicillactone A was used as a negative control. The final concentrations were 30, 10, and 3 μg/mL sorbicillactone A and dihydrosorbicillactone A. Four parallel settings of each sample were analysed. The cells were incubated for 4 hours at 37° C. After the incubation, the culture plate was centrifuged at 200×9 (˜1200 rpm) for 10 min at room temperature. The supernatant was carefully pipetted off, and the cells were each overlayered with 200 μl lysis-buffer (Roche-Kit). Then, the cells were lysed for 30 min at room temperature. After the lysis, the culture plate was again centrifuged at 200×g (˜1200 rpm) for 10 min at room temperature.

ELISA: Two strips with eight wells each were taken from the streptavidin-ELISA-plate of the Roche-kit. 20 μl of the supernatant of a cell-lysis (see above) were added into each well. In doing so, the samples with 30 or 10 μg/mL of sorbicillactone A and dihydrosorbicillactone A were loaded as 4-fold, and all other as duplicate determinations. In addition to these sample, a positive control (DNA-histone-complex) was loaded. Then, 80 μl of immune reagent were added into each well. This solution consisted of incubation buffer, anti-histone-biotin, and anti-DNA-peroxidase. The incubation buffer was used as a blank value. The ELISA-plate was incubated for 2 hours in a shaking incubator at room temperature. Them the supernatants were carefully removed, and the wells were each washed 3× with 250 μl of the incubation buffer. After the washing, 100 μl of ABTS-solution were added into each “well”. Subsequently, the cells were incubated for 30 min in the dark at room temperature. The measurement took place in a multiscan at an emission wavelength of 405 nm.

Results: The addition of 3 μg/mL of sorbicillactone A to the cells (Table 2) after 4 hours of incubation did not result in an increase of apoptosis-cells. The values 91.2% are in the range of the negative-control (=100%) (Table 2).

TABLE 2

| % of the control | MW ± SD | |
|---|---|---|
| (negative) | (OD) | sorbicillactone A |
| 322.1% | 0.219 ± 0.024 | 30 μg/mL |
| 283.8% | 0.193 ± 0.010 | 10 μg/mL |
| 91.2% | 0.062 ± 0.004 | 3 μg/mL |
| 4,641.2% | 3.156 ± 0.286 | positive control |
| 100.0% | 0.068 ± 0.005 | negative control |

After the incubation (4 hours) of the L5178Y-cells with 10 and 30 μg/mL of sorbicillactone A, the values significantly increased to 283.8% and 322.1% (p<0.001, Table 2). In doing so, the negative control was assumed as 100%.

TABLE 3

| % of the control | MW ± SD | |
|---|---|---|
| (negative) | (OD) | dihydrosorbicillactone A |
| 76.2% | 0.016 ± 0.004 | 30 μg/mL |
| 61.9% | 0.013 ± 0.002 | 10 μg/mL |
| 76.2% | 0.016 ± 0.003 | 3 μg/mL |
| 13,952.4% | 2.930 ± 0.245 | positive control |
| 100.0% | 0.021 ± 0.002 | negative control |

The addition of 3, 10, and 30 μg/mL of dihydrosorbicillactone A did not result in an induction of apoptosis in L5178Y-cells after 4 hours of incubation (Table 3). The values were reduced to 62 to 67%, below the control region.

Conclusions: Sorbicillactone A in L5178Y-cells induces apoptosis after 4 hours of incubation at a concentration of 10 and 30 μg/mL. Due to these characteristics, sorbicillactone A can be used in the treatment of leukaemia. Under the experimental conditions as chosen, dihydrosorbicillactone A did not induce apoptosis in L5178Y-cells.

1. A method for producing sorbicillactone A and/or a derivative thereof, comprising the steps of:
a) culturing a fungus of the genus Penicillium at 20-25° C. in a suitable growth medium at a salt concentration of 2-5% until the formation of a compact surface mycelium,
b) increasing the temperature to 28-35° C. and further incubation for 5-10 days,
c) separating the culture broth from the mycelium, and
d) extracting sorbicillactone A and/or a derivative thereof from the culture medium, and optionally,
e) underlaying the mycelium with fresh medium with a reduced salt concentration of 0.5-1.5% and incubation at 28-35° C. for 3-8 days,
f) repeating step c) and d), and optionally,
g) repeating steps e) to f), and
h) extracting sorbicillactone A and/or a derivative thereof from the culture medium and/or the mycelia.

2. The method according to claim 1, wherein the fungus is Penicillium chrysogenum.

3. The method according to claim 1, wherein one or more additives selected from the group consisting of pyruvate, glutamate, proline, acetate, sorbicilline and other biosynthetic precursors of sorbicillactone A is/are added to the growth media.

4. The method according to claim 1, wherein the production takes place in a flat bed method.

5. The method according to claim 1, wherein the inoculum is a solid-state-bound form of the fungus.

6. The method according to claim 5, wherein the solid state to which the fungus is bound is a floatable solid state.

7. The method according to claim 1, wherein a carrier device for a stabilisation of the surface mycelium is introduced into the culture vessel.

8. The method according to claim 7, wherein the carrier device is a mesh.

9. The method according to claim 1, wherein sorbicillactone A and/or a derivative thereof are extracted from the fungal mycelium that is separated from the culture medium by the addition of ethyl acetate.

10. The method according to claim 1, wherein sorbicillactone A and/or a derivative thereof is bound from the culture medium to a solid exchanger, and purified further from this bound form.

11. The method according to claim 10, wherein the solid exchanger is the exchange resin Amberlite XAD-16.

12. The method according to claim 10, wherein the solid exchanger as loaded is filtered off from the medium, and sorbicillactone A and/or a derivative thereof is eluted with organic solvents.

13. The method according to claim 12, which utilizes one or more organic solvents selected from the group consisting of methanol, ethanol, ethyl acetate, heptane and acetonitrile.

14. The method according to claim 10, wherein sorbicillactone A and/or a derivative thereof is acid-extracted from the crude extract with one or more organic solvents.

15. The method according to claim 14, wherein the crude extract is brought to a pH of 2 with phosphoric acid, and is subsequently extracted with ethyl acetate.

16. The method according to claim 1, wherein a purification of the extracts occurs by means of FCPC (Fast Centrifugal Partitioning Chromatography).

17. The method according to claim 16, comprising the use of a mixture of solvents from heptane, ethyl acetate, methanol, and water with an addition of 1 ml/L of concentrated phosphoric acid at a flow of 6-7 mL/min, and number of revolutions of 1200 revolutions per min, and wherein the upper is used as stationary phase.

18. The method according to claim 1, wherein a purification of the extract occurs by gel chromatography on Sephadex LH-20 using an organic solvent.

19. The method according to claim 18, wherein sorbicillactone A is eluated with methanol.

20. A method for producing sorbicillactone-A-methyl ester, comprising the steps of:
a) producing sorbicillactone A, by a method comprising the steps of:
i) culturing a fungus of the genus Penicillium at 20-25° C. in a suitable growth medium at a salt concentration of 2-5% until the formation of a compact surface mycelium,
ii) increasing the temperature to 28-35° C. and further incubation for 5-10 days,
iii) separating the culture broth from the mycelium, and
iv) extracting sorbicillactone A from the culture medium, and optionally,
v) underlaying the mycelium with fresh medium with a reduced salt concentration of 0.5-1.5% and incubation at 28-35° C. for 3-8 days,
vi) repeating step c) and d), and optionally,
vii) repeating steps e) to f), and
viii) extracting sorbicillactone A from the culture medium and/or the mycelia,
b) treating sorbicillactone A dissolved in methanol with concentrated sulphuric acid,
c) stirring at room temperature for 6 h,
d) adding water,
e) extracting with ethyl acetate,
f) evaporating the organic phases in vacuo, and
g) purifying the residual by preparative HPLC.

21. A method for producing a pharmaceutical composition, comprising the steps of:
a) producing sorbicillactone A and/or a derivative thereof by a method comprising the steps of:
i) culturing a fungus of the genus Penicillium at 20-25° C. in a suitable growth medium at a salt concentration of 2-5% until the formation of a compact surface mycelium,
ii) increasing the temperature to 28-35° C. and further incubation for 5-10 days,
iii) separating the culture broth from the mycelium, and
iv) extracting sorbicillactone A and/or a derivative thereof from the culture medium, and optionally,
v) underlaying the mycelium with fresh medium with a reduced salt concentration of 0.5-1.5% and incubation at 28-35° C. for 3-8 days,
vi) repeating step iii) and iv), and optionally,
vii) repeating steps v) to vi), and
viii) extracting sorbicillactone A and/or a derivative thereof from the culture medium and/or the mycelia, and
b) formulating a pharmaceutical composition by combining the sorbicillactone A and/or a derivative thereof obtained in step a) with pharmaceutically acceptable auxiliary agents and additives.

22. The method for producing a pharmaceutical according to claim 21, characterized in that sorbicillactone A and/or a derivative thereof is present in an amount, so that a concentration between 0.3 and 30 μg/ml is present upon treatment in vivo.

23. A method for triggering apoptosis in diseased cells; or treating leukaemia, neurodegenerative diseases, and/or bacterial or fungal infections, wherein said method comprises administering sorbicillactone A of derivatives and/or a derivative thereof.

24. The method, according to claim 23, for the treatment of leukaemia.

25. The method, according to claim 23, for the treatment of a neurodegenerative disease.

26. The method, according to claim 23, for the treatment of bacterial and fungal infections.

27. A fungal strain of the genus Penicillium chrysogenum KIP 3201 with the deposit number DSM 16137.

28. The method, according to claim 2, wherein said fungus is strain KIP 3201.
